(19) United States
(12) Patent Application Publication
(10) Pub. No.: US 2013/0123737 A1
(43) Pub. Date: May 16, 2013

(54) Wearing Article

(71) Applicant: Kaiyo Nakajima, Kagawa (JP)

(72) Inventor: Kaiyo Nakajima, Kagawa (JP)

(73) Assignee: UNI-CHARM CORPORATION, Ehrime (JP)

(21) Appl. No.: 13/260,316

(22) PCT Filed: Mar. 25, 2010
(86) PCT No.: PCT/JP10/02142

§ 371 Date: Sep. 25, 2011

(30) Foreign Application Priority Data
Mar. 25, 2009 (JP) 2009-075199

(51) Int. Cl. A61F 13/49

(52) U.S. Cl. CPC A61F 13/49019
USPC 604/385.24

(57) ABSTRACT

To alleviate any irritation experienced by the wearer's skin and/or to restrict body waste leakage in a wearing article, a sheet lying at least in a first waist region of a chassis is joined to the chassis in first and second joined regions extending in a transverse direction and opposed to each other in a longitudinal direction. Additionally or alternatively, the sheet is joined to the chassis and a liquid-absorbent structure in a pair of longitudinal joined regions respectively extending along the side edges of the liquid-absorbent structure and opposed to each other in the transverse direction. The sheet is elastic and the chassis is non-elastic, and vice versa.

### TECHNICAL FIELD

The present disclosure relates to wearing articles such as disposable diapers, toilet-training pants or incontinent briefs.

### BACKGROUND

Wearing articles each having a chassis formed of a combination of an elastic sheet and an inelastic sheet are known. For example, JP 2007-312951 A discloses a diaper that comprises an elastic outer sheet, an inelastic inner sheet and a liquid-absorbent core attached to a skin-facing side of the inner sheet. The outer sheet is folded back along a waist-opening toward the skin-facing side of the inner sheet to form a turn-back region. More specifically, the turn-back region is formed by folding the outer sheet onto the inner sheet and overlaps front and rear ends of the liquid-absorbent core. The turn-back region is bonded to these front and rear ends of the liquid-absorbent core substantially over a full width of the liquid-absorbent core.

When the front and rear ends of the liquid-absorbent core are bonded substantially over the full width of the liquid-absorbent core to the turn-back region of the outer sheet, these ends necessarily exhibit a high stiffness due to the adhesive and such high stiffness may noticeably irritate the wearer's skin, causing so-called skin trouble. Additionally, since the rest of the outer sheet is not bonded to the liquid-absorbent core in this diaper, this non-bonded region of the outer sheet can move separately from the inner sheet and elastically stretch. Consequently, by holding such inner and outer sheets in the vicinity of the waist-opening with fingers and pulling them upward, it may be difficult to pull the diaper upward to a desired position to put the diaper in close contact with the wearer's crotch, causing body waste such as urine, to leak.

It is desirable to provide a wearing article improved to alleviate any irritation experienced by the wearer's skin, on the one hand, and to restrict a possibility that leakage of bodily fluids might occur, on the other hand.

### CITATION LIST

### Patent Literature

[PTL 1]
Japanese Patent Application Laid-Open Publication No. 2007-312951 A

### SUMMARY

One or more embodiment of the present invention relates to a wearing article comprising a chassis having a longitudinal direction, a transverse direction, a first waist region, a second waist region and a crotch region extending in the longitudinal direction between the first and second waist regions, the first and second waist regions defining together a waist-opening; a liquid-absorbent structure extending in the longitudinal direction at least in the crotch region; a first sheet defining a base sheet of the chassis; and a second sheet which is laminated with the first sheet at least in transversely opposite side zones of at least the first waist region of the first and second waist regions, and is joined to the first sheet in a pair of transverse joined regions respectively extending along a pair of ends of the second sheet, the pair of ends of the second sheet extending in the transverse direction respectively on a waist-opening side and a crotch side, wherein one of the first and second sheets is elastic and the other is non-elastic.

One or more embodiment of the present invention relates to a wearing article, comprising a chassis having a longitudinal direction, a transverse direction, a first waist region, a second waist region and a crotch region extending in the longitudinal direction between the first and second waist regions, the first and second waist regions defining together a waist-opening; a liquid-absorbent structure extending in the longitudinal direction at least in the crotch region; a first sheet defining a base sheet of the chassis; and a pair of second sheets each of which is laminated with the first sheet in a respective transversely opposite side zone of at least the first waist region of the first and second waist regions, and is joined to the first sheet in a pair of transverse joined regions respectively extending along a pair of ends of the second sheet, the pair of ends of the second sheet extending in the transverse direction respectively on a waist-opening side and a crotch side, wherein one of the first and second sheets is elastic and the other is non-elastic.

One or more embodiment of the present invention relates to a wearing article comprising a chassis having a longitudinal direction, a transverse direction, a first waist region, a second waist region and a crotch region extending in the longitudinal direction between the first and second waist regions, the first and second waist regions defining together a waist-opening; a first sheet defining a base sheet of the chassis; a second sheet laminated with the first sheet at least in the first waist region of the first and second waist regions, and having a pair of ends extending in the transverse direction respectively on a waist-opening side and a crotch side, wherein one of the first and second sheets is elastic and other is non-elastic; a liquid-absorbent structure extending in the longitudinal direction at least in the crotch region and having first and second ends extending in the transverse direction and a pair of side edges extending in the longitudinal directions, wherein the first end overlaps the second sheet between the pair of ends of the second sheet; and at least a pair of longitudinal joined regions extending along the side edges of the liquid-absorbent structure wherein, in the pair of longitudinal joined regions, the liquid-absorbent structure and the first sheet are integrally joined with the second sheet.

One or more embodiment of the present invention relates to a wearing article, comprising:

a chassis having a longitudinal direction, a transverse direction, a first waist region, a second waist region and a crotch region extending in the longitudinal direction between the first and second waist regions, the first and second waist regions defining together a waist-opening; a first sheet defining a base sheet of the chassis; a pair of second sheets each of which is laminated with the first sheet in a respective transversely opposite side zone of at least the first waist region of the first and second waist regions, and having a pair of ends extending in the transverse direction respectively on a waist-opening side and a crotch side, wherein one of the first and second sheets is elastic and other is non-elastic; a liquid-absorbent structure extending in the longitudinal direction at least in the crotch region, and having first and second ends extending in the transverse direction and a pair of side edges extending in the longitudinal directions, wherein the first end overlaps the second sheet between the pair of ends of the second sheet; and at least a pair of longitudinal joined regions extending along the side edges of the liquid-absorbent structure, wherein, in the pair of longitudinal joined regions, the liquid-absorbent structure and the first sheet are integrally joined with the second sheets.

### BRIEF DESCRIPTION OF DRAWINGS

FIG. 1 is a partially cutaway perspective view of a diaper according to one embodiment of the present invention.

FIG. 2 is a partially cutaway plan view of the diaper of FIG. 1 in its flatly developed state.

FIG. 3 is an enlarged sectional view taken along the line III-III in FIG. 2.

FIG. 4 is a partial view similar to FIG. 2, showing another embodiment.

FIG. 5 is a partial view similar to FIG. 2, showing a further embodiment.

FIG. 6 is a view similar to FIG. 2, showing still another embodiment.

FIG. 7 is a view similar to FIG. 2, showing yet another embodiment.

### DETAILED DESCRIPTION

Details of wearing articles according to exemplary embodiments of the present invention will be more fully understood from the description given hereunder in reference to the accompanying drawings. It should be appreciated that same or similar reference numerals used in the specification designate same or similar members or regions through all specifically disclosed embodiments.

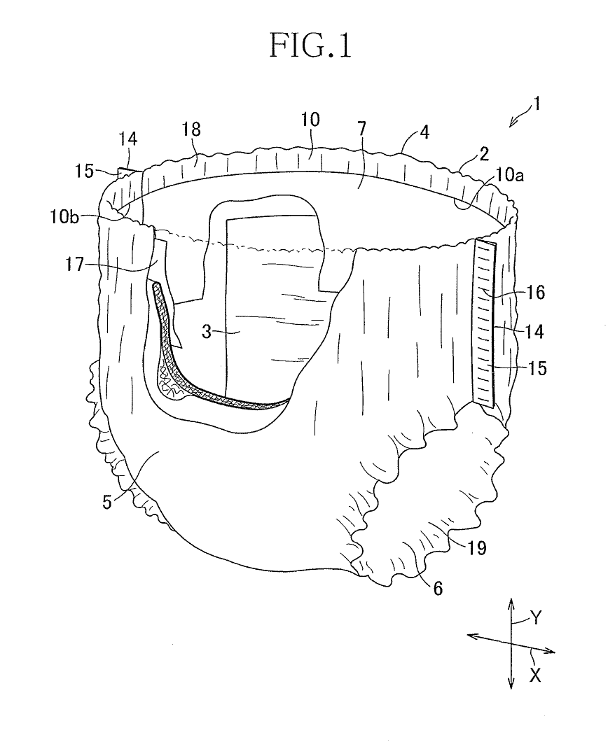

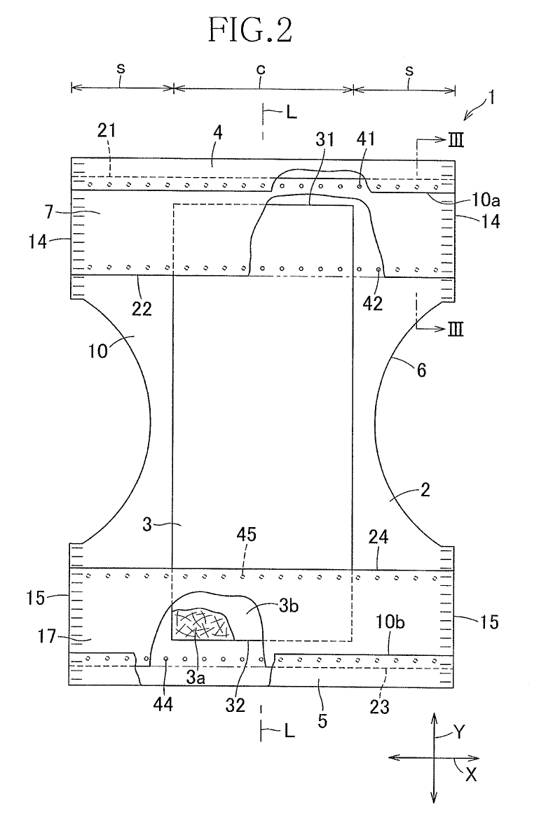

A diaper 1 in accordance with the first embodiment of the present invention is illustrated by FIGS. 1 and 2. FIG. 1 is a partially cutaway perspective view of the diaper 1, e.g., a pant-type disposable diaper, and FIG. 2 is a partially cutaway plan view of the diaper 1 flatly developed after its first and second waist regions have been disjoined from each other along respective lateral edges of these waist regions.

As seen in FIGS. 1 and 2, the diaper 1 comprises a chassis 2 and a liquid-absorbent structure 3. The chassis 2 comprises the first waist region 4, the second waist region 5 and a crotch region 6 extending between these first and second waist regions 4, 5. A direction extending from the first waist region 4 across the crotch region 6 to the second waist region 5 is referred to herein as a longitudinal direction Y, and a direction extending orthogonally to the longitudinal direction Y is referred to herein as a transverse direction X. The diaper 1 is made symmetrically about a longitudinal center line L bisecting a dimension of the diaper 1 in the transverse direction X.

With the diaper 1 being put on the wearer's body, the first waist region 4 lies on the front side of the wearer's lower torso and the second waist region 5 lies on the back side of the wearer's lower torso so that the first waist region 4 serves as the front waist region and the second waist region 5 serves as the rear waist region. With the diaper 1 being put on the wearer's body, the chassis 2 has a skin-facing side and an opposite side (also referred to herein as a garment-facing side) of the skin-facing side.

The first waist region 4 has a pair of lateral edges 14, 14 opposed to each other in the transverse direction X, and the second waist region 5 has a pair of lateral edges 15, 15 opposed to each other in the transverse direction X. The lateral edges 14, 14 are put flat and joined together with the associated lateral edges 15, 15 to form seams 16. Thereupon, the first and second waist regions 4, 5 cooperate with each other to define a waist-opening 18, while the first and second waist regions 4, 5 and the crotch region 6 cooperate together to define a pair of leg-openings 19.

The chassis 2 includes a base sheet 10 which is referred to as “first sheet” herein and formed of a non-elastic sheet. The liquid-absorbent structure 3 comprises an absorbent core 3a wrapped with a liquid-pervious sheet 3b, extending from the first waist region 4 across the crotch region 6 to the second waist region 5, and attached to the base sheet 10 on its skin-facing side. The liquid-absorbent structure 3 is contoured by the first and second ends 31, 32 lying in the first and second waist regions 4, 5, respectively, and transversely opposite side edges (unnumbered).

The chassis 2 further includes an elastic first waist sheet 7 and an elastic second waist sheet 17 in the first and second waist regions 4, 5, respectively. Each or both of the first waist sheet 7 and the second waist sheet 17 is/are referred to as “second sheet” herein. These first and second waist sheets 7, 17 are elastic both in the longitudinal direction Y and in the transverse direction X and respectively laminated with the base sheet 10.

The first waist sheet 7 has a pair of ends, i.e., first and second ends 21, 22, extending in the transverse direction X and opposed to each other in the longitudinal direction Y. As viewed in the longitudinal direction Y, the first end 21 lies on the waist-opening side between the peripheral edge of the waist-opening 18 and the first end 31 of the liquid-absorbent structure 3, and the second end 22 lies on the crotch side and is placed from the first end 31 of the liquid-absorbent structure 3 toward the crotch region 6. Along these first and second ends 21, 22, there are respectively provided a pair of transverse joined regions, i.e., first and second joined regions 41, 42, each comprising a plurality of joints intermittently arranged in the transverse direction X. The first waist sheet 7 is attached in a stretched state in the transverse direction X to the base sheet 10 in the first and second joined regions 41, 42 so that the first waist sheet 7 is elastically contractible. In a part of the second joined region 42 where the liquid-absorbent structure 3 is sandwiched between the base sheet 10 and the first waist sheet 7, the first waist sheet 7 is attached not to the base sheet 10 but to the liquid-absorbent structure 3.

Similarly, the second waist sheet 17 has a pair of ends, i.e., third and fourth ends 23, 24, extending in the transverse direction X and opposed to each other in the longitudinal direction Y. As viewed in the longitudinal direction Y, the third end 23 lies on the waist-opening side between the peripheral edge of the waist-opening 18 and the second end 32 of the liquid-absorbent structure 3, and the fourth end 24 lies on the crotch side and is placed from the second end 32 of the liquid-absorbent structure 3 toward the crotch region 6. Along these third and fourth ends 23, 24, there are respectively provided fourth and fifth joined regions 44, 45 each comprising a plurality of joints intermittently arranged in the transverse direction X. The second waist sheet 17 is attached in a stretched state in the transverse direction X to the base sheet 10 in the fourth and fifth joined regions 44, 45 so that the second waist sheet 17 is elastically contractible. In a part of the fifth joined region 45 wherein the liquid-absorbent structure 3 is sandwiched between the base sheet 10 and the second waist sheet 17, the second waist sheet 17 is attached not to the base sheet 10 but to the liquid-absorbent structure 3.

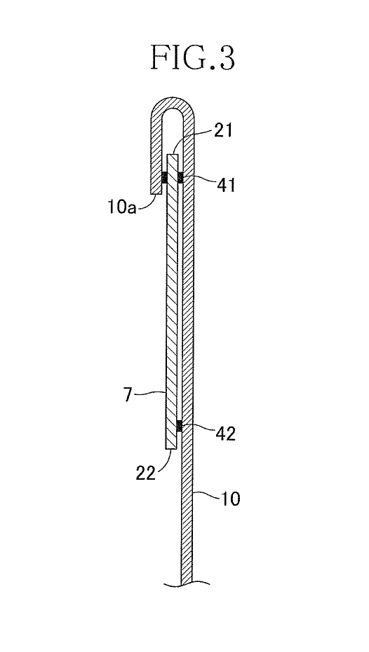

Referring to FIG. 3, a first end 10a of the base sheet 10 is folded along the peripheral edge of the waist-opening 18 back onto the first waist sheet 7 and bonded to both surfaces of the first waist sheet 7 in the transverse joined region 41. A second end 10b of the base sheet 10 is folded along the peripheral edge of the waist-opening 18 back onto the second waist sheet 17 and bonded thereto (See FIG. 2).

The first and second waist sheets 7, 17 may be elastically stretched in the transverse direction X to expand the waist-opening 18 to the maximum dimension and thereby to facilitate the diaper 1 to be put on the wearer's body. The first and second waist sheets 7, 17 may be then left to elastically contract in the transverse direction X to keep the waist-opening 18 of the diaper 1 in close contact with the wearer's waist.

The first and second waist sheets 7, 17 are joined not in a stretched state in the longitudinal direction Y to the base sheet 10 formed of a non-elastic sheet. As a consequence, the first and second waist sheets 7, 17 are restricted from being elastically stretched and contracted in the longitudinal direction Y between the first joined region 41 and the second joined region 42, and between the fourth joined region 44 and the fifth joined region 45, respectively.

The diaper 1 according to this embodiment is intended to be put on the wearer's body by pulling the diaper 1 upward with the first waist region 4 and/or the second waist region 5 held by the fingers in the vicinity of the waist-opening 18. The above-described unique arrangement for the elasticity restriction in the longitudinal direction Y facilitates the diaper 1 to be pulled upward to the predetermined position.

When both the first waist region 4 and the second waist region 5 are held by the fingers in the vicinity of the waist-opening 18 in order to put the diaper 1 on the wearer's body, the elasticity restriction in the longitudinal direction Y is preferably assured both in the first waist sheet 7 and the second waist sheet 17. However, it is also possible without departing from the scope of the invention to provide such elasticity restriction in only one of the first and second waist sheets 7, 17, for example, only in the first waist sheet 7. One embodiment may be contemplated in which the second waist sheet 17 is fixed to the base sheet 10 only in the fourth joined region 44, and the fifth joined region 45 is not provided. In this embodiment, the elasticity restriction in the longitudinal direction Y is not provided in the second waist sheet 17.

The respective joined regions in which the first and second waist sheets 7, 17 are fixed to the base sheet 10 may be defined in continuous fashion but preferably defined in intermittent fashion. Expression “the joined region(s) is/are intermittently defined” used herein means that two or more sheets are joined together in intermittently arranged small-area regions, also referred to as “joints” herein, each having an appropriate shape such as a dot or a line-segment.

A joined region, in which two or more sheets are joined together, necessarily exhibits relatively high stiffness and has a harsh texture. If such joined region exhibiting high stiffness continuously extends in the direction, in which one or more of the elastic sheets should be elastically contractible, such continuous joined region might disturb the expected elasticity of the one or more sheet. This problem may be effectively overcome by the intermittently arranged joints wherein, preferably, a distance between each pair of the adjacent joints is sufficiently closed up to avoid an undesirable situation that the wearer's toe might be caught between the adjacent joints when the diaper is put on the wearer's body. This step of joining is preferably carried out by, but not limited to, an embossing treatment under heating. For example, it is also possible to carry out this joining step using other means, such as heat seal or ultrasonic seal.

A dimension of a region defined between the peripheral edge of the waist-opening 18 and the first and second waist sheets 7, 17, respectively, in the longitudinal direction Y is preferably in a range of 20 mm to 40 mm. This is the region supposed to be subject to the thumb's force when the diaper 1 is held in the vicinity of the waist-opening 18 with the fingers to put the diaper 1 on the wearer's body. In this way, the diaper 1 can be smoothly put on the wearer's body without stretching the chassis 2 in the longitudinal direction Y. This is for the reason that the thumb's force is exerted on the region in which the base sheet 10 is put flat and joined together with the first and second waist sheets 7, 17, respectively, when the diaper 1 is held by the fingers in the vicinity of the waist-opening 18 and pulled up.

The diaper 1 has, as viewed in the transverse direction X, a central zone c in which the liquid-absorbent structure 3 and the first waist sheet 7 or the second waist sheets 17 overlap each other, and opposite side zones s extending on both sides of the central zone c. The absorbent core constituting the liquid-absorbent structure 3 usually contains super-absorbent polymer particles or fluff pulp to enhance liquid-absorption capacity and dispersing ability of the liquid-absorbent structure 3 and correspondingly has stiffness higher than that of the first and second waist sheets 7, 17. As a result, the elasticity of the first and second waist sheets 7, 17 is significantly restricted by the absorbent core. However, the first and second waist sheets 7, 17 do not overlap the liquid-absorbent structure 3 in the side zones s, and therefore, the elasticity of the first and second waist sheets 7, 17 is assured in the side zones s. In one or more embodiments disclosed herein where the fifth joined region 45 is not provided, the elasticity of the second waist sheet 17 is not affected by the absorbent core.

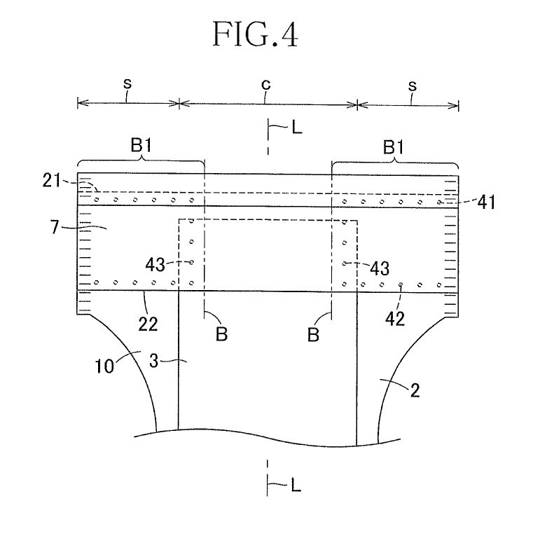

A diaper in accordance with the second embodiment of the present invention is illustrated by FIG. 4.

The diaper according to the second embodiment is similar to the diaper illustrated by FIGS. 1 through 3 except that the joined regions provided in the central zone c of the first waist region 4 are distinguished from those in FIGS. 1 through 3. Referring to FIG. 4, the first and second joined regions 41, 42 are provided only in the side zones s and the first waist sheet 7 is joined to the base sheet in these first and second joined regions 41, 42. In the central zone c, a pair of longitudinal joined regions, i.e. third joined regions 43 are provided along the side edges of the liquid-absorbent structure 3 to extend in the longitudinal direction Y and, in these third joined regions 43, the base sheet 10 and the liquid-absorbent structure 3 are integrally joined with the first waist sheet 7. This step of joining is preferably carried out by, but not limited to, an embossing treatment under heating. For example, it is also possible to carry out this joining step using other means such as heat seal or ultrasonic seal.

The base sheet 10 and the liquid-absorbent structure 3 are integrated in these third joined regions 43 with the first waist sheet 7 so that the base sheet 10 and the liquid-absorbent structure 3 may smoothly move in a cooperative fashion as the diaper is pulled up on the wearer's body.

While the central zone c is formed with two rows of the third joined regions 43 each extending only along the side edges of the liquid-absorbent structure 3 in the illustrated second embodiment, it is also possible to provide the central zone c with one or more additional rows of the joined regions 43 extending in parallel to the two rows, for example, a row of the joined regions 43 extending along the longitudinal center line L. The third joined regions 43 may be formed in the continuous fashion or in the intermittent fashion.

It is not essential for the first waist sheet 7 and/or the second waist sheet 17 to extend fully in the transverse direction X as illustrated, but the desired effect can be achieved as long as the first waist sheet 7 and/or the second waist sheet 17 is/are provided in opposite lateral zones B1 extending outward from imaginary lines B extending in the longitudinal direction Y in the region occupied by the liquid-absorbent structure 3 as indicated by FIG. 4. In other words, it is possible to replace, e.g., the first waist sheet 7, with two smaller waist sheets in the regions B1 and with no waist sheet in the region between the lines B. Each of the smaller waist sheets is bonded at the first and second joined regions 41, 42 to the base sheet 10, and at the third joined regions 43 to the liquid-absorbent structure 3.

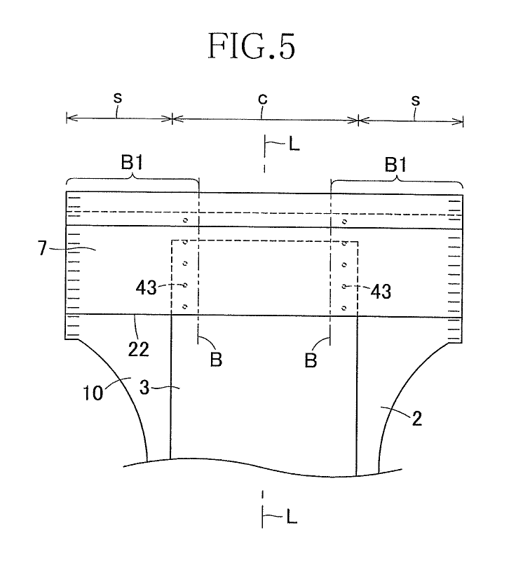

In an alternative embodiment shown in FIG. 5, the first and second joined regions 41, 42 are not essential and have been omitted.

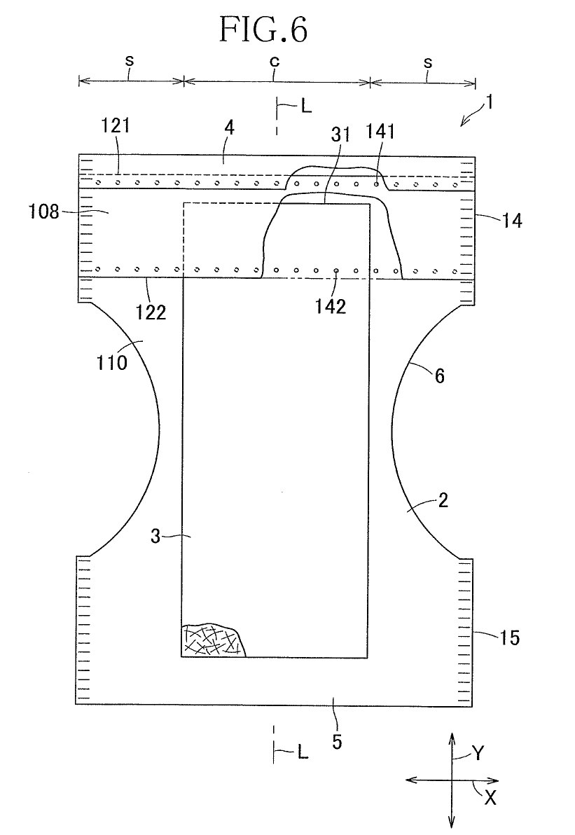

A diaper in accordance with the third embodiment of the present invention is illustrated by FIG. 6.

As apparent from FIG. 6, the diaper 1 according to the third embodiment is similar to the diaper 1 according to the first embodiment except that, in contrast with the first embodiment, an elastic sheet is used as the base sheet and a non-elastic sheet is used as the first and/or second waist sheet(s). More specifically, the base sheet 110 of the chassis 2 is formed of an elastic sheet having elasticity in the longitudinal direction Y as well as in the transverse direction X. The chassis 2 further includes, in the first waist region 4, the non-elastic first waist sheet 108 laminated with the base sheet 110. The assembly of the base sheet 110 and the first waist sheet 108 has, the central zone c in which the sheet 108 overlaps the liquid-absorbent structure 3, and the side zones extending outward from the central zone c as viewed in the transverse direction X.

Referring to FIG. 6, the first waist sheet 108 has first and second ends 121, 122 both extending in the transverse direction X. In the longitudinal direction Y, the first end 121 lies between the peripheral edge of the waist-opening and the first end 31 of the liquid-absorbent structure 3, and the second end 122 is placed from the first end 31 toward the crotch region 6. Along these first and second ends 121, 122, first and second joined regions 141, 142 each comprising a plurality of joints intermittently formed are provided, respectively. It should be appreciated here that these first and second joined regions 141, 142 will effectively function also when these first and second joined regions 141, 142 are continuously formed, respectively. In these first and second joined regions 141, 142, the first waist sheet 108 is joined to the elastic base sheet 110, when the base sheet 110 is under tension in the transverse direction X, so that the base sheet 110 is elastically contractible. In the second joined region 142 in the central zone c, the liquid-absorbent structure 3 is sandwiched between the base sheet 110 and the first waist sheet 108 and the first waist sheet 108 is joined not to the base sheet 110 but to the liquid-absorbent structure 3.

The base sheet 110 formed of an the elastic sheet assures the diaper 1 as a whole to be put in close contact with the wearer's skin. In addition, this elastic base sheet covers the liquid-absorbent structure 3 from the opposite side of the skin-facing side to press the liquid-absorbent structure 3 against the wearer's skin and thereby to assure high fitness.

The first waist sheet 108 is joined to the base sheet 110, when the base sheet 110 is in a non-stretched state in the longitudinal direction Y. As a consequence, elasticity of the base sheet 110 in the longitudinal direction Y is restricted between the first joined region 141 and the second joined region 142. Such unique arrangement for the elasticity restriction in the longitudinal direction Y advantageously facilitates the diaper 1 to be put on the wearer's body by pulling the diaper 1 upward to the predetermined position with the first waist region 4 held by the fingers in the vicinity of the waist-opening.

When the second waist region 5 is intended to be held by the fingers in the vicinity of the waist-opening 18 in order to put the diaper 1 on the wearer's body, it is possible to join a non-elastic second waist sheet (not shown) to the base sheet 110 also in the second waist region 5 similarly to the non-elastic first waist sheet 108 in the first waist region 4.

In the third embodiment, the first waist sheet 108 provided on the skin-facing side of the first waist region 4 is formed with gathers as the elastic base sheet 110 contracts in the transverse direction X, and these gathers may irritate the wearer's skin. To avoid this, an additional elastic sheet (not shown) may be laminated on the skin-facing side of the first waist sheet 108 to sandwich the first waist sheet 108 as a whole between the elastic base sheet 110 and this additional elastic sheet. Such additional elastic sheet is joined to the elastic base sheet 110 and the first waist sheet 108 along the first and second ends 121, 122.

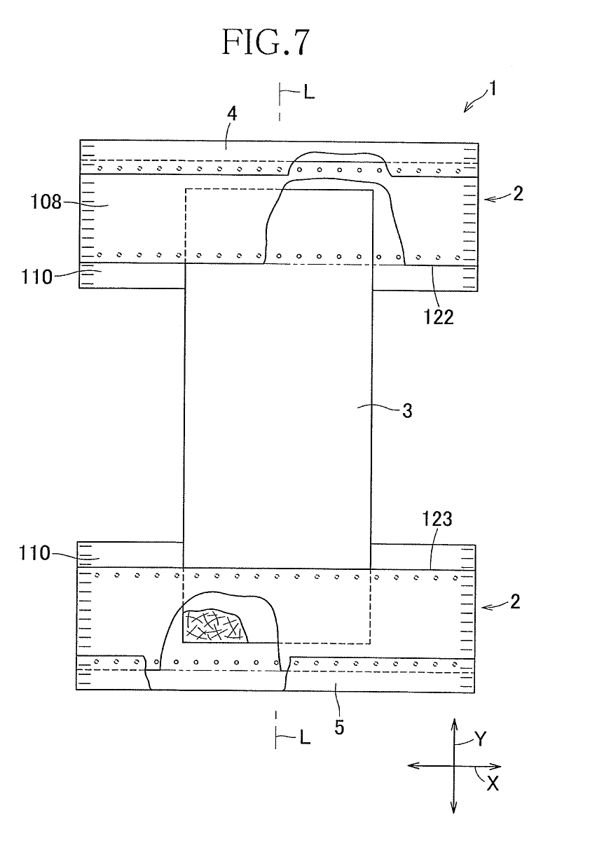

As a variant of the third embodiment shown in FIG. 7, the base sheet 110 may be initially segmented into the first waist region 4 and the second waist region 5 and these segments may be connected one to another by the liquid-absorbent structure 3, which defines the crotch region. Such variant is convenient to stretch the sheet (s) defining the first waist region 4 and/or the second waist region 5 in the transverse direction X.

In the first and second embodiments, the first waist sheet lies closer to the wearer's skin compared to the liquid-absorbent structure 3, i.e., the first end 31 of the liquid-absorbent structure 3 is sandwiched between the first waist sheet 7 and the base sheet 10. In the third embodiment also, the first end 31 of the liquid-absorbent structure 3 is sandwiched between the first waist sheet 108 and the base sheet 110. Such construction is advantageous for the purpose of preventing the absorbent core from falling off the front end of the liquid-absorbent structure 3. However, one or more embodiments of the present invention may be implemented in the form of a diaper in which the liquid-absorbent structure 3 lies closer to the wearer's skin compared to the elastic first waist sheet 7 or the non-elastic first waist sheet 108. In such embodiment(s), it is possible to provide an additional sheet on the side of the liquid-absorbent structure 3 facing the wearer's skin so as to cover the front end of the liquid-absorbent structure 3.

While the first and second waist sheets 7, 17 in the first and second embodiments as well as the first waist sheet 108 in the third embodiment lie closer to the wearer's skin compared to the base sheet 10/110, it is also possible to join these sheets to the base sheet on the opposite side of the skin-facing side.

While the elastic sheet used in disclosed embodiments of the present invention may include an elastically stretchable and contractible plastic film, an elastic fibrous non-woven fabric is preferably used in consideration of comfortable texture experienced by the wearer. In some embodiments, such elastic non-woven fabric is made from (A) an elastically stretchable non-woven fabric made of elastic fibers, such as urethane elastic fibers, or (B) an elastically stretchable non-woven fabric made of elastically stretchable fibers mixed with non-elastically stretchable thermoplastic synthetic fibers. In other embodiments, a plurality of rubber strings can be used as the leg elastic members.

Sheet material for the base sheet is preferably or liquid-impervious. For example, a laminate of two or more hydrophobic fibrous non-woven fabric layers or a laminate of such hydrophobic fibrous non-woven fabric(s) and a plastic film can be used as the base sheet.

While the diaper according to one or more of embodiments of the present invention exemplarily described above with respect to the first, second and third embodiments comprises the chassis entirely defined by the elastic or non-elastic base sheet and the liquid-absorbent structure attached to the skin-facing side of this base sheet, the present invention is not limited to such construction. For example, the present invention may be implemented in one or more embodiments as a diaper comprising a base sheet formed of a laminate of a liquid-pervious top-sheet facing the wearer's skin and a liquid-impervious back-sheet facing away from the wearer's skin and the liquid-absorbent structure sandwiched between these top- and back-sheets.

As stock materials used to implement one or more embodiments of the present invention, those commonly used for relevant articles such as disposable diapers and menstruation napkins may be used. For example, a fibrous non-woven fabric or a perforated plastic film as the liquid-pervious sheet; a moisture-pervious but liquid-impervious plastic film or a laminate of a moisture-pervious but liquid-impervious plastic film and a fibrous non-woven fabric as the liquid-impervious sheet; a mixture of fluff pulp and super-absorbent polymer particles and optionally thermoplastic staple fibers wrapped with a liquid-absorbent and dispersant sheet, such as tissue paper, as the absorbent structure; and a natural or synthetic rubber or an elastic fibrous non-woven fabric as elastic members, e.g., for the leg openings 19.

According to one or more embodiments of the present invention, joining of the diaper components may be carried out by means usually used in the relevant technical field, for example, bonding by adhesive, such as hot melt adhesive, and sealing, such as heat sealing or ultrasonic sealing particularly for thermoplastic sheets.

While the absorbent articles according to embodiments of the present invention have been exemplarily described to be pants-type disposable diapers, further embodiments of the present invention may be implemented also in the form of so-called open-type diapers provided on the lateral edges of the first and second waist regions with releasable fastening means, such as mechanical or adhesive fasteners, instead of the seams of the pants-type diapers.

The aspects of the present invention described above may be arranged in at least the following items:

(i) A wearing article (1), comprising:

a chassis (2) having a longitudinal direction (Y), a transverse direction (X), a first waist region (4), a second waist region (5) and a crotch region (6) extending in the longitudinal direction between the first and second waist regions, the first and second waist regions defining together a waist-opening (18);

a liquid-absorbent structure (3) extending in the longitudinal direction at least in the crotch region;

a first sheet (10) defining a base sheet of the chassis; and

a second sheet (7) which is laminated with the first sheet of at least the first waist region of the first and second waist regions, and is joined to the first sheet in a pair of transverse joined regions (41, 42) respectively extending along a pair of ends (21, 22) of the second sheet, the pair of ends of the second sheet extending in the transverse direction respectively on a waist-opening side and a crotch side;

wherein one of the first and second sheets is elastic and the other is non-elastic.

(ii) A wearing article (1), comprising:

a chassis (2) having a longitudinal direction (Y), a transverse direction (X), a first waist region (4), a second waist region (5) and a crotch region (6) extending in the longitudinal direction between the first and second waist regions, the first and second waist regions defining together a waist-opening (18);

a liquid-absorbent structure (3) extending in the longitudinal direction at least in the crotch region;

a first sheet (10) defining a base sheet of the chassis; and

a pair of second sheets (7) each of which is laminated with the first sheet in a respective transversely opposite side zone of at least the first waist region of the first and second waist regions, and is joined to the first sheet in a pair of transverse joined regions (41, 42) respectively extending along a pair of ends of the second sheet, the pair of ends (21, 22) of the second sheet extending in the transverse direction respectively on a waist-opening side and a crotch side, wherein one of the first and second sheets is elastic and the other is non-elastic.

(vi) A wearing article (1), comprising:

a chassis (2) having a longitudinal direction (Y), a transverse direction (X), a first waist region (4), a second waist region (5) and a crotch region (6) extending in the longitudinal direction between the first and second waist regions, the first and second waist regions defining together a waist-opening (18);

a first sheet (10) defining a base sheet of the chassis;

a second sheet (7) laminated with the first sheet at least in the first waist region of the first and second waist regions, and having a pair of ends (21, 22) extending in the transverse direction respectively on a waist-opening side and a crotch side, wherein one of the first and second sheets is elastic and other is non-elastic;

a liquid-absorbent structure (3) extending in the longitudinal direction at least in the crotch region and having first and second ends (31, 32) extending in the transverse direction and a pair of side edges extending in the longitudinal directions, wherein the first end overlaps the second sheet between the pair of ends of the second sheet; and

at least a pair of longitudinal joined regions (43) extending along the side edges of the liquid-absorbent structure, wherein, in the pair of longitudinal joined regions, the liquid-absorbent structure and the first sheet are integrally joined with the second sheet.

(vii) A wearing article (1), comprising:

a chassis (2) having a longitudinal direction (Y), a transverse direction (X), a first waist region (4), a second waist region (5) and a crotch region (6) extending in the longitudinal direction between the first and second waist regions, the first and second waist regions defining together a waist-opening (18);

a first sheet (10) defining a base sheet of the chassis;

a pair of second sheets (7) each of which is laminated with the first sheet in a respective transversely opposite side zone of at least the first waist region of the first and second waist regions, and having a pair of ends (21, 22) extending in the transverse direction respectively on a waist-opening side and a crotch side, wherein one of the first and second sheets is elastic and other is non-elastic;

a liquid-absorbent structure (3) extending in the longitudinal direction at least in the crotch region, and having first and second ends (31, 32) extending in the transverse direction and a pair of side edges extending in the longitudinal directions, wherein the first end overlaps the second sheet between the pair of ends of the second sheet; and

at least a pair of longitudinal joined regions (43) extending along the side edges of the liquid-absorbent structure, wherein, in the pair of longitudinal joined regions, the liquid-absorbent structure and the first sheet are integrally joined with the second sheets.

The aspect of the present invention described in the above items (i) and (ii) may provide one or more of the following advantageous effects.

(a) In this wearing article, an elastic sheet and a non-elastic sheet are laminated on and joined to each other at least in a first waist region in a pair of transverse joined regions extending in a transverse direction. With this construction, the elastic sheet is able to be elastically contracted in the transverse direction but its elastic stretch in the longitudinal direction is substantially restricted by the non-elastic sheet so that the diaper can be smoothly pulled upward to a predetermined position when the diaper is put on the wearer's body. The elastic sheet is joined to the non-elastic sheet not over a large area but along the joined regions. Consequently, the desired texture of the sheets can be substantially maintained and the wearer's skin can be protected against so-called skin trouble.

The aspect of the present invention described in the above items (vi) and (vii) may provide one or more of the following advantageous effects.

(b) In this wearing article, one of the elastic sheet and the non-elastic sheet and the liquid-absorbent structure are integrally joined with the other sheet in a pair of the longitudinal joined regions respectively extending along the side edges of the liquid-absorbent structure and opposed to each other in the transverse direction. Therefore, it is assured that the liquid-absorbent structure is cooperatively pulled upward with the sheet(s) when the sheet(s) is/are held by fingers and pulled upward on the wearer's body.

Additionally, one or more of the following embodiments of the present invention described in the above items (i) and (ii) are provided in accordance with further aspects.

(iii) The liquid-absorbent structure has first and second ends (31, 32) extending in the transverse direction and a pair of side edges extending in the longitudinal directions, wherein the first end overlaps the second sheet between the pair of ends of the second sheet.

(iv) The wearing article further including a pair of longitudinal joined regions (43) extending along the side edges of the liquid-absorbent structure in the longitudinal direction, wherein, in the pair of longitudinal joined regions, the liquid-absorbent structure and the second sheet are integrally joined together.

(v) The first sheet is folded along a peripheral edge of the waist-opening back toward the crotch region to sandwich the end of the second sheet on the waist-opening side, and is bonded to both surfaces of the second sheet in the transverse joined region provided on the waist-opening side.

According to the embodiments in the above (iii)-(v), one or more of the advantageous effect(s) set forth in (a) are better ensured, as well as one or more of the advantageous effects of the respective embodiments that have previously been referred to in the respective related descriptions are ensured.

Additionally, one or more of the following embodiments of the aspects of the present invention described in the above items (i), (ii), (vi) and (vii) are provided in accordance with further aspects.

(viii) The joined regions comprise a plurality of intermittently arranged joints.

(ix) The second sheet is continuously laminated with the first sheet in the transverse direction.

(x) The first sheet and the second sheet sandwich the first end of the liquid-absorbent structure therebetween.

(xi) The wearing article further comprises another second sheet (17) which is laminated with the first sheet at least in transversely opposite side zones of the second waist region, has a pair of ends (23, 24) extending in the transverse direction respectively on a waist-opening side and a crotch side, and is joined to the first sheet in at least one further transverse joined region (44) extending along at least the end (23) on the waist-opening side.

(xii) The another second sheet is joined to the first sheet in a pair of the transverse joined regions (44, 45) extending along both ends (23, 24) of the another second sheet, respectively.

(xiii) The wearing article further comprises another second sheet (17) laminated with the first sheet at least in transversely opposite side zones of the second waist region, has a pair of ends (23, 24) extending in the transverse direction respectively on a waist-opening side and a crotch side, and is joined to the liquid-absorbent structure integrally in at least a pair of further longitudinal joined regions extending along the side edges of the liquid-absorbent structure.

(xiv) The second sheets is elastic and the first sheet is non-elastic.

(xv) The first sheets is elastic and the second sheet is non-elastic.

(xvi) The first sheet comprises separate segments (110, FIG. 7) defining the first waist region and the second waist region, and the liquid-absorbent structure defines the crotch region and connects the segments of the first sheet in the longitudinal direction.

(xvii) In at least the first waist region, a dimension in the longitudinal direction between a peripheral edge of the waist-opening and the end of the second waist sheet on the waist-opening side is ranging from 20 mm to 40 mm.

(xviii) The wearing article further comprises another longitudinal joined region provided between the pair of longitudinal joined regions, wherein, in the another longitudinal joined region, the liquid-absorbent structure and the second sheet are integrally joined together.

(xix) The wearing article further comprises an elastic sheet laminated to the second, non-elastic sheet for covering and preventing gathers, which are formed in the second, non-elastic sheet when the first, elastic sheet elastically contracts, from contacting a wearer's skin, in use.

(xx) The second sheet(s) is joined only in the joined regions.

According to the embodiments in the above (viii)-(xx), one or more of the advantageous effects set forth in (a) and (b) are better ensured, as well as one or more of the advantageous effects of the respective embodiments that have previously been referred to in the respective related descriptions are ensured.

The terms “first”, “second”, “third”, “fourth” and “fifth” herein are use merely for distinguishing between similar elements. Furthermore, the term “first waist region” means one of the front and rear waist regions, and the term “second waist region” means the other.

1. A wearing article, comprising:
a chassis having a longitudinal direction, a transverse direction, a first waist region, a second waist region and a crotch region extending in said longitudinal direction between said first and second waist regions, said first and second waist regions defining together a waist-opening;
a liquid-absorbent structure extending in said longitudinal direction at least in said crotch region;
a first sheet defining a base sheet of said chassis; and
a second sheet which is laminated with said first sheet of at least said first waist region of said first and second waist regions, and is joined to said first sheet in a pair of transverse joined regions respectively extending along a pair of ends of said second sheet, said pair of ends of said second sheet extending in said transverse direction respectively on a waist-opening side and a crotch side,
wherein one of said first and second sheets is elastic and the other is non-elastic.

2. A wearing article, comprising:
a chassis having a longitudinal direction, a transverse direction, a first waist region, a second waist region and a crotch region extending in said longitudinal direction between said first and second waist regions, said first and second waist regions defining together a waist-opening;
a liquid-absorbent structure extending in said longitudinal direction at least in said crotch region;
a first sheet defining a base sheet of said chassis; and
a pair of second sheets each of which is laminated with said first sheet in a respective transversely opposite side zone of at least said first waist region of said first and second waist regions, and is joined to said first sheet in a pair of transverse joined regions respectively extending along a pair of ends of said second sheet, said pair of ends of said second sheet extending in said transverse direction respectively on a waist-opening side and a crotch side,
wherein one of said first and second sheets is elastic and the other is non-elastic.

3. The wearing article defined by claim 1, wherein
said liquid-absorbent structure has first and second ends extending in said transverse direction and a pair of side edges extending in said longitudinal directions, and
said first end overlaps said second sheet between said pair of ends of said second sheet.

4. The wearing article defined by claim 3, further comprising
at least a pair of longitudinal joined regions extending along said side edges of said liquid-absorbent structure in said longitudinal direction,
wherein, in said pair of longitudinal joined regions, said liquid-absorbent structure and said second sheet are integrally joined together.

5. The wearing article defined by claim 1, wherein
said first sheet is folded along a peripheral edge of said waist-opening back toward said crotch region to sandwich said end of said second sheet on said waist-opening side, and is bonded to both surfaces of said second sheet in said transverse joined region provided on said waist-opening side.

6. A wearing article, comprising:
a chassis having a longitudinal direction, a transverse direction, a first waist region, a second waist region and a crotch region extending in said longitudinal direction between said first and second waist regions, said first and second waist regions defining together a waist-opening;
a first sheet defining a base sheet of said chassis;
a second sheet laminated with said first sheet at least in said first waist region of said first and second waist regions, and having a pair of ends extending in said transverse direction respectively on a waist-opening side and a crotch side, wherein one of said first and second sheets is elastic and other is non-elastic;
a liquid-absorbent structure extending in said longitudinal direction at least in said crotch region, and having first and second ends extending in said transverse direction and a pair of side edges extending in said longitudinal directions, wherein said first end overlaps said second sheet between said pair of ends of said second sheet; and
at least a pair of longitudinal joined regions extending along said side edges of said liquid-absorbent structure, wherein, in said pair of longitudinal joined regions, said liquid-absorbent structure and said first sheet are integrally joined with said second sheet.

7. A wearing article, comprising:
a chassis having a longitudinal direction, a transverse direction, a first waist region, a second waist region and a crotch region extending in said longitudinal direction between said first and second waist regions, said first and second waist regions defining together a waist-opening;
a first sheet defining a base sheet of said chassis;
a pair of second sheets each of which is laminated with said first sheet in a respective transversely opposite side zone of at least said first waist region of said first and second waist regions, and having a pair of ends extending in said transverse direction respectively on a waist-opening side and a crotch side, wherein one of said first and second sheets is elastic and other is non-elastic;
a liquid-absorbent structure extending in said longitudinal direction at least in said crotch region, and having first and second ends extending in said transverse direction and a pair of side edges extending in said longitudinal directions, wherein said first end overlaps said second sheet between said pair of ends of said second sheet; and
at least a pair of longitudinal joined regions extending along said side edges of said liquid-absorbent structure, wherein, in said pair of longitudinal joined regions, said liquid-absorbent structure and said first sheet are integrally joined with said second sheets.

8. The wearing article defined by claim 1, wherein said joined regions comprise a plurality of intermittently arranged joints.

9. The wearing article defined by claim 1, wherein
said second sheet is continuously laminated with said first sheet in said transverse direction.

10. The wearing article defined by claim 3, wherein
said first sheet and said second sheet sandwich said first end of said liquid-absorbent structure therebetween.

11. The wearing article defined by claim 1, further comprising
another second sheet which is laminated with said first sheet at least in transversely opposite side zones of said second waist region, has a pair of ends extending in said transverse direction respectively on a waist-opening side and a crotch side, and is joined to said first sheet in at least one further transverse joined region extending along at least said end on said waist-opening side.

12. The wearing article defined by claim 11, wherein
said another second sheet is joined to said first sheet in a pair of said transverse joined regions extending along both ends of said another second sheet, respectively.

13. The wearing article defined by claim 1 further comprising
another second sheet which is laminated with said first sheet at least in transversely opposite side zones of said second waist region, has a pair of ends extending in said transverse direction respectively on a waist-opening side and a crotch side, and is joined to said liquid-absorbent structure integrally in at least a pair of further longitudinal joined regions extending along said side edges of said liquid-absorbent structure.

14. The wearing article defined by claim 1, wherein
said second sheets is elastic and said first sheet is non-elastic.

15. The wearing article defined by claim 1, wherein
said first sheets is elastic and said second sheet is non-elastic.

16. The wearing article defined by claim 1, wherein
said first sheet comprises separate segments defining said first waist region and said second waist region, and
said liquid-absorbent structure defines said crotch region and connects said segments of said first sheet in said longitudinal direction.

17. The wearing article defined by claim 1, wherein
in at least said first waist region, a dimension in said longitudinal direction between a peripheral edge of said waist-opening and said end of said second waist sheet on said waist-opening side is ranging from 20 mm to 40 mm.

18. The wearing article defined by claim 4 further comprising
another longitudinal joined region provided between said pair of longitudinal joined regions,
wherein, in said another longitudinal joined region, said liquid-absorbent structure and said second sheet are integrally joined together.

19. The wearing article defined by claim 15, further comprising
an elastic sheet laminated to said second, non-elastic sheet for covering and preventing gathers, which are formed in said second, non-elastic sheet when said first, elastic sheet elastically contracts, from contacting a wearer's skin, in use.

20. The wearing article as claimed in claim 1, wherein said second sheet(s) is joined only in said joined regions.
